(19) United States
(12) Patent Application Publication
(10) Pub. No.: US 2024/0093254 A1
(43) Pub. Date: Mar. 21, 2024

(54) METHOD FOR INCREASING PRODUCTIVITY OF 2'-FUCOSYLLACTOSE THROUGH CHANGES IN CULTURE MEDIUM COMPOSITION AND CULTURING

(71) Applicants: Advanced Protein Technologies Corp., Suwon-si, Gyeonggi-do (KR); SEOUL NATIONAL UNIVERSITY R&DB FORNDATION, Seoul (KR)

(72) Inventors: Chul Soo SHIN, Suwon-si (KR); Jong Won YOON, Seongnam-si (KR); Young Ha SONG, Yongin-si (KR); Young Sun YOO, Hwaseong-si (KR); Jeong Su BANG, Ansan-si (KR); Heon Hak LEE, Pyeongtaek-si (KR)

(73) Assignees: Advanced Protein Technologies Corp., Suwon-si, Gyeonggi-do (KR); SEOUL NATIONAL UNIVERSITY R&DB FORNDATION, Seoul (KR)

(21) Appl. No.: 18/286,832

(22) PCT Filed: Oct. 31, 2022
(86) PCT No.: PCT/KR2022/016816

§ 371 Date: Oct. 13, 2023

(30) Foreign Application Priority Data
Nov. 3, 2021 (KR) 10-2021-0149938

(51) Int. Cl. C12P 19/12; C12N 9/10; C12N 9/12; C12N 15/72; C12N 15/77

(52) U.S. Cl. CPC C12P 19/12; C12N 9/1051; C12N 9/1241; C12N 15/72; C12N 15/77; C12Y 204/01069; C12Y 207/07013; C12N 2800/101

(57) ABSTRACT

The present invention relates to a method for increasing the productivity of 2′-fucosyllactose through various changes in culture medium composition and culturing on the basis of lactose, which is a substrate, wherein 2-fucosyllactose can be continuously produced in a high-yield at an optimum lactose concentration discovered by a culturing method of the present invention.

### TECHNICAL FIELD

The present invention relates to a method for improving the productivity of 2′-fucosyllactose (2′-FL) through changes in culture medium composition and culture method, and more specifically, to a method for improving the productivity of 2′-fucosyllactose (2′-FL) through changes in culture method and culture medium composition based on a lactose substrate.

### BACKGROUND ART

Human milk oligosaccharides (HMOs) are oligosaccharides contained in human milk, which are the third most abundant component after lactose and fat. There are about 200 types of a variety of human milk oligosaccharides. Human milk oligosaccharides have advantages of strengthening the immune function or having positive effects on the development and behaviors of children.

2′-fucosyllactose, which is present in the largest amount in major HMOs, is involved in various biological activities. Methods for preparing 2′-fucosyllactose reported by previous research include direct extraction from breast milk and extraction based on chemical or enzymatic treatment. However, direct extraction from breast milk has problems in that it is unethical, breast milk supply is limited and productivity is low. In addition, the chemical synthesis method has problems such as expensive substrates, low isomer selectivity and yield, the necessity of use of toxic reagents and high purification costs and the enzymatic synthesis method has problems in that GDP-L-fucose used as a precursor is very expensive and the purification cost of the fucose transferase is high.

As one approach to address these problems, 2′-fucosyllactose may be prepared using microorganisms. However, most conventional methods of preparing 2′-fucosyllactose have used recombinant E. coli. However, it is recognized as a harmful germ by consumers and E. coli cells are limitedly used due to a phenomenon called “lactose killing” in which E. coli cells may be killed under lactose-restricted culture by lactose permease (Daniel Dykhuizen and Daniel Hartl, 1987, “Transport by the lactose permease of Escherichia coli as the basis of lactose killing”, 10.1128/JB.135.3.876-882, 1978, Journal of Bacteriology). Accordingly, there is a need for a novel method for preparing 2-fucosyllactose to improve the productivity in a safe and economically efficient manner, while overcoming the drawbacks of conventional methods.

### DISCLOSURE

### Technical Problem

It is an object of the present invention to provide a method for improving the productivity of 2′-fucosyllactose (2′-FL) through changes in a culture method and a culture medium using a concentration of lactose as a substrate.

### Technical Solution

In accordance with an aspect of the present invention, the above and other objects can be accomplished by the provision of a method for preparing 2′-fucosyllactose by culturing recombinant Corynebacterium glutamicum in a medium supplemented with lactose, wherein the recombinant Corynebacterium glutamicum is transformed to express α-1,2-fucosyltransferase, is transformed to express GDP-D-mannose-4,6-dehydratase, is transformed to express GDP-L-fucose synthase, and is transformed to express lactose permease, and the Corynebacterium glutamicum has phosphomannomutase and GTP-mannose-1-phosphate guanylyltransferase, wherein the culture is performed while the lactose is maintained at a concentration of 30 to 150 g/L.

Preferably, the recombinant Corynebacterium glutamicum may be transformed to overexpress phosphomannomutase, and is transformed to overexpress GTP-mannose-1-phosphate guanylyltransferase.

Preferably, the medium may further contain glucose.

Preferably, the method may be performed by fed-batch culture of further feeding glucose or lactose during culture.

### Advantageous Effects

In the present invention, the optimal lactose concentration to increase the amount of 2′-fucosyllactose produced was determined by the following experiment. In addition, by adding lactase at the late stage of culture, guanosine diphosphate-L-fucose, a precursor of 2′-fucosyllactose, was produced using glucose produced by the degradation of lactose, and reaction with undegraded lactose is induced, so that the lactose remaining at the late stage of culture could be utilized to the fullest extent for the production of 2′-fucosyllactose.

### DESCRIPTION OF DRAWINGS

FIG. 1 is a schematic diagram illustrating a process of preparing 2′-fucosyllactose using a recombinant Corynebacterium strain;

FIG. 2 is a graph showing the productivity of 2′-fucosyllactose depending on a lactose concentration;

FIG. 3 is a schematic diagram illustrating the degradation of lactose using beta-galactosidase; and

FIG. 4 is a graph showing the productivity of 2′-fucosyllactose through lactose degradation.

### BEST MODE

2′-fucosyllactose, which is a main ingredient of human milk oligosaccharides, has health functional advantages such as being involved in various biological activities, and various methods for producing the same have been developed. However, direct extraction from breast milk and chemical or enzymatic synthesis have problems such as low productivity, high costs, low production yield, and toxicity. Therefore, there is a need for alternatives thereto. For this purpose, production using microorganisms has been proposed, but most thereof used recombinant E. coli. The use of E. coli cells is limited due to the phenomenon, called “lactose killing”, in which E. coli cells are killed by the action of lactose permease. Accordingly, the present invention aims at optimizing the culture medium composition and culture method to produce a large amount of 2′-fucosyllactose using lactose, as a substrate, and providing a method for safely and efficiently producing 2′-fucosyllactose even though it exhibits a certain level of lactose killing.

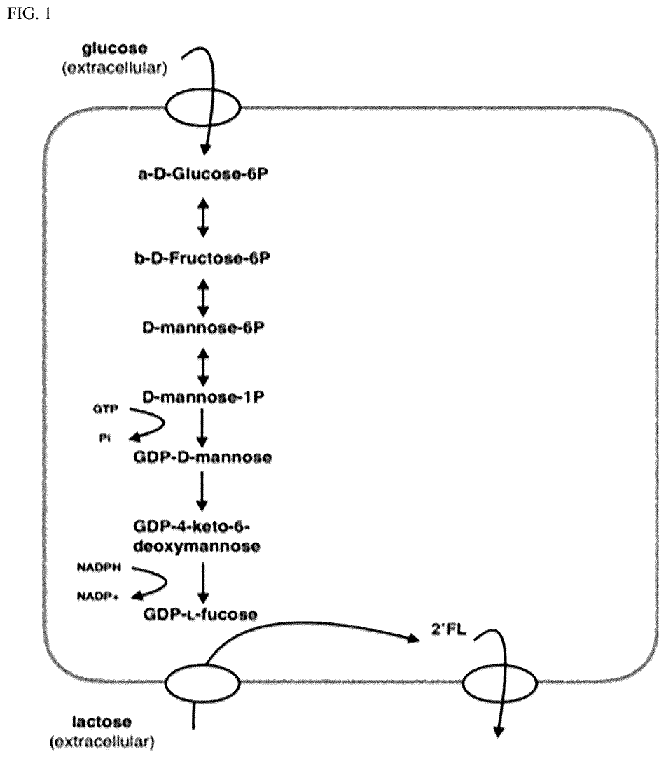

Therefore, in one aspect, the present invention is directed to a method for preparing 2′-fucosyllactose by culturing recombinant Corynebacterium glutamicum in a medium supplemented with lactose, wherein the recombinant Corynebacterium glutamicum is transformed to express α-1,2-fucosyltransferase, is transformed to express GDP-D-mannose-4,6-dehydratase, is transformed to express GDP-L-fucose synthase, and is transformed to express lactose permease, and the Corynebacterium glutamicum has phosphomannomutase and GTP-mannose-1-phosphate guanylyltransferase, wherein the culture is performed while the lactose is maintained at a concentration of 30 to 150 g/L. The process for preparing 2′-fucosyllactose using the strain of the present invention is shown in FIG. 1.

The present inventors suggested a method of preparing 2′-FL using recombinant Corynebacterium glutamicum in previous Korean Patent Nos. 10-1731263 (registered on Apr. 24, 2017) and 10-2014925 (registered on Aug. 21, 2019). In the prior art, in order to optimally produce 2′-fucosyllactose, culture was performed while the amount of glucose that was supplied was set as a rate-limiting factor and an initial concentration of the lactose was set at 10 g/L. However, in the present invention, based on the idea that the amount of lactose that was supplied could be a rate-limiting factor, it could be from the result of the following experiment that, when the lactose concentration was increased, the production of 2′-fucosyllactose increased in a concentration-dependent manner.

In the present invention, it was found that 2′-fucosyllactose can be produced in high yield by culturing recombinant Corynebacterium microorganisms at a lactose concentration of 30 to 150 g/L. More preferably, it was found that 2′-fucosyllactose could be produced in high yield by culturing recombinant Corynebacterium microorganisms at a lactose concentration of 40 to 100 g/L. The lactose concentration for optimal production of 2′-fucosyllactose was set at 40 to 100 g/L by which the ranges showing significant differences from the immediately preceding experimental group or immediately following experimental group, among the experimental groups exhibiting 2′-fucosyllactose productivity of 40 g/L or more, were set as the lower limit (40 g/L) and upper limit (100 g/L), respectively.

Meanwhile, in the method of preparing 2′-fucosyllactose according to the present invention, the recombinant Corynebacterium glutamicum is preferably transformed to overexpress phosphomannomutase, and is transformed to overexpress GTP-mannose-1-phosphate guanylyltransferase. Since Corynebacterium glutamicum has its own genes encoding phosphomannomutase (ManB) and GTP-mannose-1-phosphate guanylyltransferase (ManC), and thus can express the same, it is not necessary to incorporate the genes encoding these enzymes, but it is necessary to overexpress the enzymes for mass production. Therefore, in the present invention, preferably, it is necessary to transform Corynebacterium glutamicum to overexpress the two enzymes.

Meanwhile, the term “expression” as used herein means incorporation and expression of external genes into strains in order to intentionally express enzymes that cannot be inherently expressed by the Corynebacterium glutamicum strain according to the present invention, and the term “overexpression” as used herein means overexpression that is induced by artificially increasing the amount of expressed enzyme in order to increase expression for mass-production, although the Corynebacterium glutamicum strain according to the present invention has genes encoding the corresponding enzyme and therefore can self-express the same.

Meanwhile, regarding the method for preparing 2′-fucosyllactose according to the present invention, the medium preferably further includes glucose. By adding an additional ingredient to the medium, the growth of strains can be facilitated and 2′-fucosyllactose can thus be produced at higher productivity.

Meanwhile, in the method of preparing 2′-fucosyllactose of the present invention, the method is preferably fed-batch culture including further supplying glucose or lactose. When glucose or lactose is continuously fed through fed-batch culture, the growth of the cells can be further increased, and 2′-fucosyllactose can be produced with high purity, high yield, and high productivity. The detailed technologies associated with fed-batch culture are well-known in the art and are not described herein.

Meanwhile, in the method of preparing 2′-fucosyllactose of the present invention, it is preferable to add a lactase to the medium in the latter part of the stationary phase of the culture. At this time, the lactase is preferably beta-galactosidase. The lactose is degraded to produce galactose and glucose. GDP-L-fucose, which is the final substrate for the synthesis of 2′-fucosyllactose, is produced from the produced glucose, and then reacted with remaining undigested lactose to further produce 2′-fucosyllactose. Through this process, the yield of 2′-fucosyllactose can be increased by making the most of lactose, which remains as a by-product in the late stage of fermentation.

Hereinafter, the present invention will be described in more detail with reference to the following examples, but the scope of the present invention is not limited to the examples, and includes variations and technical concepts equivalent thereto.

### Preparation Example 1: Preparation of Recombinant Plasmids

Escherichia coli K-12 MG1655 and Corynebacterium glutamicum ATCC 13032 were used in order to produce plasmids and 2′-fucosyllactose (2′-FL), respectively.

In order to establish pFGW(Ps) plasmids, gmd-wcaG gene clusters were amplified through PCR reaction using two DNA primers, namely GW-F and GW-R, from the genomic DNAs of K-12 MG1655, E. Coli, the promoters of the Sod gene were amplified through PCR reaction using two DNA primers, namely Sod-F and Sod-R from the genomic DNA of Corynebacterium glutamicum ATCC 13032, and then PSod-Gmd-WcaG DNA fragments were synthesized through an overlapping PCR reaction using two DNA primers, namely Sod-F and GW-R.

In addition, the transcription termination sequence was amplified from the pXMJ19 plasmids through PCR reaction using two DNA primers, namely Ter-F and Ter-R, and a PSod-Gmd-WcaG-ter sequence was synthesized from the synthesized pSod-Gmd-WcaG and transcription termination sequence as templates through PCR reaction using DNA primers Sod-F and Ter-R, and was then inserted into the pCES208 plasmids sieved by the restriction enzyme, BamHI, to establish pGW plasmids.

In addition, a Tuf gene promoter was amplified through PCR reaction using two DNA primers Tuf-F1 and Tuf-R1 from the genomic DNAs of Corynebacterium glutamicum ATCC 13032, and α-1,2-fucosyltransferase was amplified through PCR reaction using two DNA primers, FT(Ps)-F and FT(Ps)-R, from the synthesized α-1,2-fucosyltransferase derived from Pseudopedobacter saltans DSM 12145, and pTuf-FT (Ps) DNA fragments were synthesized through an overlapping PCR reaction using two primers Tuf-F and FT(Ps)-R. The pTuf-FT (Ps) DNA fragments were inserted into the established pGW plasmid by treating with restriction enzyme NotI to establish PFGW(Ps) plasmids.

Meanwhile, in order to establish pXIL plasmids, lacY genes were amplified through PCR reaction using two DNA primers, namely ilvC-lacY-F and lacY pX-R, from the genomic DNAs of K-12 MG1655, E. Coli, the promoters of the ilvC genes were amplified through PCR reaction using two DNA primers, namely pX-ilvC-F and ilvC-lacY-R, from the genomic DNA of Corynebacterium glutamicum ATCC 13032, pilvC-lacY DNA fragments were synthesized through an overlapping PCR reaction using two DNA primers, namely pX-ilvC-F and ilvC-lacY-R, and the pilvC-lacY fragments were inserted into the pX plasmid (pXMJ19) treated with restriction enzymes, Not I and EcoR I to establish pXIL plasmids.

The strains, primers, plasmids, and nucleic acid and amino acid sequences used in this Preparation Example are shown in Tables 1 to 4 below.

TABLE 1
Strains

| | |
|---|---|
| E. Coli K-12 MG1655 | F⁻, lambda⁻, rph-1 |
| C. glutamicum | Wild-type strain, ATCC13032 |

TABLE 2
Nucleic acid and amino acid sequences

| | |
|---|---|
| gmd Nucleic acid sequence | SEQ ID NO: 1 |
| wcaG Nucleic acid sequence | SEQ ID NO: 2 |
| lacY Nucleic acid sequence | SEQ ID NO: 3 |
| FT(Ps) Nucleic acid sequence | SEQ ID NO: 4 |
| FT(Ps) Amino acid sequence | SEQ ID NO: 5 |

TABLE 3

| Primers | Sequence (5′-3′) |
|---|---|
| px-ilvC- | GTCATATGATGGTCGCGGATCCGAATTCCCAGGCAAGCTCCGC |
| F | |
| ilvC- | GTTTTTTAAATAGTACATAATCTCGCCTTTCGTAAAAATTTTGGT |
| lacY-R | |
| ilvC- | TTACGAAAGGCGAGATTATGTACTATTTAAAAAACACAAACTTTTGGAT |
| lacY-F | GTTCGG |
| lacY pX- | GCCTTTCGTTTTATTTGCTCGAGTGCGGCCGCTTAAGCGACTTCATTCA |
| R | CCTGACGAC |
| Tuf-F1 | TGGAGCTCCACCGCGGTGGCTGGCCGTTACCCTGCGAA |
| Tuf-R1 | CAAATATCATTGTATGTCCTCCTGGACTTCG |
| FT (ps)-F | AGGACATACAATGATATTTGTAACCGGATATG |
| FT (ps)-R | CGCTTCACTAGTTCTAGAGCTTAAATAATGTGTCGAAACAGATTC |
| Sod-F | TTGGAGCTCCACCGCGGTGGCGGCCGCTCTAGAACTAGTGAAGCGCCTC |
| | ATCAGCG |
| Sod-R | TACACCGGTGATGAGAGCGACTTTTGACATGGTAAAAAATCCTTTCGTA |
| | GGTTTCCGCAC |
| GW-F | ATGTCAAAAGTCGCTCTCATCACCGGTGTA |
| GW-R | CAAGCTGAATTCTTACCCCCGAAAGCGGTC |
| ter-F | GACCGCTTTCGGGGGTAAGAATTCAGCTTG |
| ter-R | GGTATCGATAAGCTTGATATCGAATTCCTGCAGCCCGGGGAAAAGGCCA |
| | TCCGTCAGGAT |

TABLE 4
Plasmids

| Plasmid | Related features | Ref. |
|---|---|---|
| pCES208 | Km^{R}, C. glutamicum/E. coli | J. Microbiol. |
| | shuttle vector | Biotechnol. (2008), |
| | | 18 (4), 639647 |
| pXMJ19 | Cm^{R}, C. glutamicum/E. coli | Biotechnology |
| | shuttle vector | Techniques (1999), |
| | | 13, 437441 |
| pGW | pCES208 + Sod-gmd-wcaG | Patent No. 10-2014925 |
| pFGW(Ps) | pCES208 + Tuf-FT(Ps) + | Patent No. 10-2014925 |
| | Sod-gmd-wcaG | |
| pXIL | pXMJ19 + ilvC-lacY | Patent No. 10-2014925 |

### Example 1: Productivity of 2′-Fucosyllactose Depending on Lactose Concentration

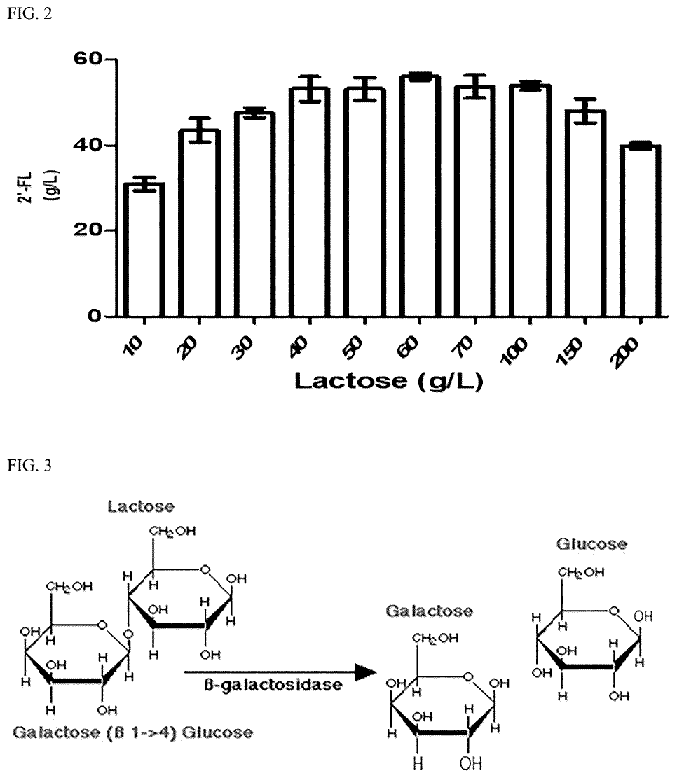

The culture was performed while the concentration of lactose remaining in the medium during the culture was maintained at 10, 20, 30, 40, 50, 60, 70, 100, 150, or 200 g/L, so that an experiment was performed while the lactose concentration was adjusted to ±5 g/L of the standard concentration. The culture was performed while the concentration of 2′-fucosyllactose produced depending on the lactose concentration was measured at each time point. The result of the culture, as shown in FIG. 2, showed that 2′-fucosyllactose was produce at the highest concentration in the range of 40 to 100 g/L.

### Example 2: Productivity of 2′-Fucosyllactose Depending on Lactase Treatment

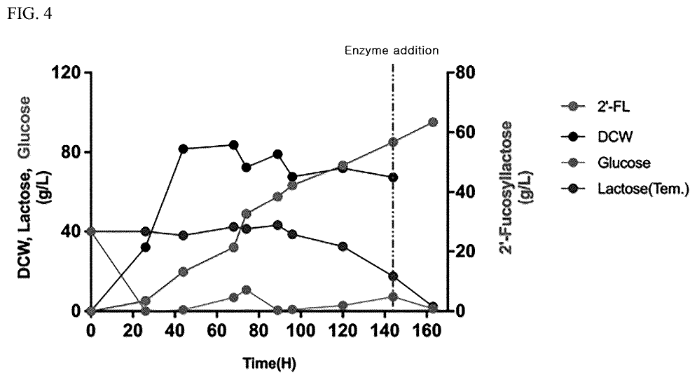

When lactose is degraded by the enzyme beta-galactosidase, glucose and galactose are produced (FIG. 3). In the latter part of the stationary phase of the culture, beta-galactosidase enzyme was added. As a result, the production of 2′-fucosyllactose increased again, as shown in FIG. 4. This result shows that GDP-L-fucose, which is a final substrate for the synthesis of 2′-fucosyllactose, is produced from glucose obtained by lactose degradation, and then is reacted with undegraded lactose to produce 2′-fucosyllactose. 2′-fucosyllactose can be further produced by maximally utilizing lactose through treatment with a lactase, which is present as a by-product in the latter part of the stationary phase of culture.

1. A method for preparing 2′-fucosyllactose at high yield by culturing recombinant Corynebacterium glutamicum in a medium supplemented with lactose,
wherein the recombinant Corynebacterium glutamicum is transformed to express α-1,2-fucosyltransferase, is transformed to express GDP-D-mannose-4,6-dehydratase, is transformed to express GDP-L-fucose synthase, and is transformed to express lactose permease, and the Corynebacterium glutamicum has phosphomannomutase and GTP-mannose-1-phosphate guanylyltransferase,
wherein the culture is performed while the lactose is maintained at a concentration of 40 to 100 g/L.

2. The method according to claim 1, wherein the recombinant Corynebacterium glutamicum is transformed to overexpress phosphomannomutase, and is transformed to overexpress GTP-mannose-1-phosphate guanylyltransferase.

3. The method according to claim 1, wherein the medium further comprises glucose.

4. The method according to claim 3, wherein the method is performed by fed-batch culture of further feeding glucose or lactose during culture.


SEQUENCE LISTING (5 sequences)

SEQ ID NO: 1 - DNA, 1122 bp, Escherichia coli
  source 1..1122: mol_type = genomic DNA
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataa

SEQ ID NO: 2 - DNA, 966 bp, Escherichia coli
  source 1..966: mol_type = genomic DNA
atgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaa

SEQ ID NO: 3 - DNA, 1254 bp, Escherichia coli
  source 1..1254: mol_type = genomic DNA
atgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaa

SEQ ID NO: 4 - DNA, 807 bp, unidentified
  source 1..807: mol_type = genomic DNA
atgatatttgtaaccggatatggccagatgtgtaacaacatccttcaatttgggcatttctttgcttatgcaaaaagaaatggtttaaaaacggttggcttacgtttttgctacaaatacacttttttcaagattagtaacgaaaaaggctataattggccgacctatctttatgcaaaatatggggcaaaaataggacttataaagtctgttgattttgacgaatcattcgaaggtacaaatgtagattctcttcaattagacaaacaaaccgtgttagccaaaggctggtattttagagactaccagggatttcttaattaccgtaatgagcttaaagcacttttcgactttaaagagcatattaagaaaccggtagaacagtttttttcaacgttatcaaaagacaccatcaaagtaggcctgcatataagacgtggtgattataagacctggcaccagggtaaatacttttttagcgacgaagaatacggtcaaatcgtaaattcttttgctaaaagtttagataaaccggtagaattaattattgttagcaatgatcccaaactaaacagcaaaagttttgaaaatttaacatcctgtaaagtatcaatgttaaatggcaatcctgccgaagatctttaccttctttctaaatgtgattatattattggccctcccagcactttttctttaatggcagctttttacgaagaccgccctttatattggatatttgataaagaaaaacagcttttagcagaaaactttgacaagttcgagaatctgtttcgacacattatttaa

SEQ ID NO: 5 - protein, 268 aa, unidentified
MIFVTGYGQMCNNILQFGHFFAYAKRNGLKTVGLRFCYKYTFFKISNEKGYNWPTYLYAKYGAKIGLIKSVDFDESFEGTNVDSLQLDKQTVLAKGWYFRDYQGFLNYRNELKALFDFKEHIKKPVEQFFSTLSKDTIKVGLHIRRGDYKTWHQGKYFFSDEEYGQIVNSFAKSLDKPVELIIVSNDPKLNSKSFENLTSCKVSMLNGNPAEDLYLLSKCDYIIGPPSTFSLMAAFYEDRPLYWIFDKEKQLLAENFDKFENLFRHII